(19) United States
(12) Patent Application Publication
(10) Pub. No.: US 2025/0082181 A1
(43) Pub. Date: Mar. 13, 2025

(54) Carrier Device for Preassembling,Holding, and Inserting an Image Sensor Assembly into a Shaft of a Medical Imaging Device, Medical Imaging Device and Method of Preassembling

(71) Applicant: KARL STORZ SE & Co KG, Tuttlingen (DE)

(72) Inventor: Johannes Eisenlauer, Rietheim-Weilheim (DE)

(73) Assignee: KARL STORZ SE & Co KG, Tuttlingen (DE)

(21) Appl. No.: 18/826,088

(22) Filed: Sep. 5, 2024

(30) Foreign Application Priority Data
Sep. 7, 2023 (DE) 102023124089.7

(51) Int. Cl. A61B 1/00; A61B 1/05

(52) U.S. Cl. CPC A61B 1/00097; A61B 1/05

(57) ABSTRACT

The invention relates to a carrier device for pre-mounting and holding a sensor assembly and for insertion into a shaft of a medical imaging device, the carrier device having as a respective side a distal side, a proximal side, a top side, a bottom side and two opposing sides, the sensor assembly being assignable to the carrier device and having at least one sensor and the sensor being mountable on the distal side of the carrier device, the carrier device having a continuous cavity from the proximal side to the distal side, so that, when the sensor is pre-mounted on the distal side of the carrier device, the sensor can be aligned from the inside into a predetermined position by means of an alignment tool which can be inserted into the cavity. Furthermore, the invention relates to a medical imaging device and a method for pre-assembling a sensor assembly.

### CROSS REFERENCE TO RELATED APPLICATIONS

This application claims priority to German Patent Application No. 102023124089.7, filed Sep. 7, 2023, and entitled, “Trägervorrichtung zum Vormontieren und Halten einer Sensorbaugruppe und zum Einsetzen in einen Schaft einer medizinischen Bildgebungsvorrichtung, medizinische Bildgebungsvorrichtung und Verfahren zum Vormontieren,” which is incorporated herein by reference.

### FIELD OF THE INVENTION

The invention relates medical imaging devices and endoscopic instruments in particular.

### BACKGROUND OF THE INVENTION

Medical imaging devices, such as exoscopes or endoscopes, are used to view an surface such as an interior surface or object of interest of a human or animal body. Endoscopic imaging devices for technical, rather than medical, applications are often referred to as borescopes and are similarly used. Medical imaging devices usually have at least one sensor and/or image sensor. Endoscopes employ an objective lens system for collecting image light and generating an image of a viewing area. The objective lens (or lens system) is usually arranged in a distal end section of a shaft of the endoscope. A video endoscope is an endoscope where the electronic image sensor for generating the endoscopic image from the collected image light is usually arranged in the distal end section of the shaft, with its sensor plane lying on an image plane of the objective lens system. Such video endoscopes are referred to as chip-on-the-tip endoscopes (COTT endoscopes).

Due to the preferred small diameter of the endoscopic shaft, usually just a few millimeters, there are increased requirements for the assembly and attachment of the objective lens system and the sensor and/or image sensor within the cavity of the shaft. Due to the limited space, especially in the distal end section of the shaft, there is a risk that the objective lens system and/or the sensor and/or image sensor will not be optimally aligned with each other or that any such misalignment, or other serious issues, will only be discovered after final assembly during factory testing of the endoscope that the sensor and/or image sensor. In addition, the sensor and/or image sensor must be fixed, for example by an adhesive, inside the shaft. This carries the disadvantage that undefined contact points can occur during the bonding, and the adhesive can spread in an undesired and uncontrolled manner due to the small component dimensions and the tight installation conditions. Additionally, there is a need for fast, simple and cost-effective manufacturing processes, particularly in the production of medical imaging devices designed for single use, such as disposable endoscopes. On the other hand, for reusable endoscopes, the assembly process must be so secure and reliable that the sensor and/or image sensor mounted in the distal end section retains its position within the shaft for the scopes extended life time, despite regular cleaning and sterilization, for example by autoclaving at high temperatures and with pressurized steam, and the adhesive bond does not come loose.

### BRIEF DESCRIPTION OF THE INVENTION

A carrier device for pre-mounting and holding a sensor assembly and for insertion into a shaft of a medical imaging device is presented. The carrier device includes a distal side, a proximal side, a top side, a bottom side and two opposing sides, which may be referred to as a left side and a right side, wherein the sensor assembly can be associated with the carrier device and that has at least one sensor and the sensor can be mounted on the distal side of the carrier device, wherein the carrier device has a continuous cavity from the proximal side to the distal side configured such that when the sensor is pre-mounted on the distal side of the carrier device, the sensor can be aligned from the inside into a predetermined position by means of an alignment tool which can be inserted into the cavity.

Thus, a carrier device is provided to which a sensor and/or a sensor assembly can be pre-assembled and/or attached outside of the medical imaging device, that is in manufacturing step and/or steps performed prior to (i.e., preassembly) the positioning of the carrier/sensor within the shaft of the medical imaging device. Consequently, after pre-assembly, the sensor assembly and/or the sensor arranged on the carrier device can be tested for functionality, before the carrier device with the pre-assembled sensor assembly and/or the sensor, is inserted into the medical imaging device, which is usually in a distal end section of a shaft. When applied to a video endoscope, the carrier device provides a preassembly that can be mounted outside the video shaft and can be tested for a functioning video image before it is installed in the video shaft.

It advantageous that the carrier device has a continuous cavity extending from its proximal side to its distal side that can be used multifunctionally during assembly and subsequently during the operation of the medical imaging device. This enables the sensor to be aligned to a predetermined position and/or spatial orientation from within the cavity during pre-assembly by means of an alignment tool inserted into the cavity. If necessary, it is also possible to readjust the alignment of the sensor after inserting the carrier device with the pre-assembled sensor assembly and/or the sensor within the distal end section, for example by using a tool bent at approximately 90° and inserting it proximally into the continuous cavity. This allows the alignment of the pre-mounted sensor to be corrected after inserting the carrier device with the pre-mounted sensor assembly into the shaft.

It is particularly advantageous that both the carrier device itself and its continuous cavity can have other functions during operation of the medical imaging device subsequent to the preassembly of the sensor assembly and insertion into the shaft. In addition to the aforementioned holding function, the carrier device itself can also be used as a heat transfer element, for example to dissipate and/or distribute operating heat generated by the sensor and/or a light source arranged on the distal side, which will aid in avoiding the overheating of the distal end section of the shaft.

It should be noted that the sensor does not necessarily have to be mounted on the distal side of the carrier device. In such embodiments, the objective and/or the objective lens system can comprise several components, which can be divided up and can, for example, be mounted with some components on the distal side and with other components on the proximal side of the carrier device insofar as the continuous cavity may ensure optical continuity through the carrier device. If the components of an objective or an objective lens system are divided between the distal and proximal sides of the carrier device, the sensor can be accordingly arranged on the proximal side of the divided objective and/or objective lens system.

An important aspect of the invention is that use of the carrier device not only enables improved pre-assembly and/or holding of the sensor assembly and/or the sensor outside of the medical imaging device, but also provides the continuous cavity previously described such that the carrier device can be used multifunctionally both during assembly and during operation of the medical imaging device. The fact that the carrier device enables preassembly and functional testing outside the medical imaging device means that the carrier device with the pre-assembled sensor assembly is easier to insert into the confined space within the shaft and can be fastened more reliably. As a result, many uses of the imaging device are possible despite repeated cleaning and sterilization. Alternatively, the production of simple, reliable, and cost-effective disposable endoscopes is enabled.

### BRIEF DESCRIPTION OF THE DRAWINGS

The invention is further explained by the following exemplary descriptions of particular embodiments, considered along with the accompanying drawings.

FIG. 1 is a schematic partial three-dimensional view of a video endoscope and a display system.

FIG. 2 is a three-dimensional representation of a sensor holder seen from its distal side.

FIG. 3 is a schematic side view of a mounted sensor assembly on the sensor holder of FIG. 2.

FIG. 4 is a schematic three-dimensional representation of the sensor assembly pre-mounted on the sensor holder inserted into a distal end section of a shaft as seen from above through a mounting opening.

FIG. 5 is a schematic three-dimensional side view of the pre-assembled and inserted sensor assembly on the sensor holder from FIG. 4 with a heat conduction tube arranged on the proximal side.

FIG. 6 is a flow diagram of steps in the method of pre-assembling a sensor assembly.

### DETAILED DESCRIPTION OF THE INVENTION

The following terms are used throughout the disclosure.

A “medical imaging device” is any type of technical and/or electrical device that is suitable for recording, processing and/or forwarding an image of a viewing area in a medical environment or in a technical environment. A medical imaging device can be, for example, an endoscope, an exoscope, or a borescope.

An “exoscope” is a medical device or instrument for observing and/or illuminating a viewing area on a patient from a position away from the patient's body. An exoscope can also be a medical microscope. An “endoscope” is a medical or industrial device for endoscopic examination and observation of a human or animal body cavity and/or an industrial cavity, such as a pipe or other enclosed space. In particular, the endoscope may include a handpiece, a shaft, a light source, a light guide, a sensor, and/or a camera. The term endoscope may include a video endoscope, which includes the ability to record and transmit a digital image. The video endoscope may be a chip-on-the-tip (COTT) endoscope, where the image sensor is, for example, a CMOS or CCD chip that is arranged within the distal end section of the shaft of the video endoscope. The image data recorded by the image sensor can be transmitted electronically through the shaft in the proximal direction to the handpiece and further to a display system and/or to an image processing unit, such as a camera control unit (CCU) in order to process, display, and/or display the endoscopic image for the user. In addition to human and veterinary applications, the endoscope or video endoscope can also be used for industrial purposes, for example for visual inspection in cavities that are difficult to access. In industrial applications, an endoscope is often referred to as a borescope.

An endoscope or exoscope as a device in itself can also be part of an overall imaging system, which can have further devices such as further sensors, measuring devices, cables, a camera control unit, a light source, a display device for displaying the captured image data, a monitor and the like.

A “shaft” is an elongated and hollow tube for at least partial insertion into a body cavity. Throughout this specification, reference will be made to the shaft of the endoscope being inserted into a body cavity, but it should be understood that this is not limiting, and the endoscope, in any of these embodiments and implementations, may be a borescope, and the borescope may be inserted into an opening in a technical or industrial material. Tshaft can be a flexible and/or rigid. For cases where the shaft is rigid, the distal end section in particular is designed to be bendable. In some cases, the shaft may have rigid and flexible regions. In particular embodiments, for example, the shaft may be rigid through the majority of its length, but it may have a flexible distal end that may be used to, for example, look around corners. The shaft in most embodiments will have a diameter in the range of 2 mm to 10 mm. In principle, the shaft can have any cross-sectional shape, however, it is generally preferable that the shaft have a round or oval cross-section. The interior of the shaft can include further components, such as an optical waveguide for directing illuminating light to an object field, a working channel or several working channels for supplying irrigation fluid or tools, such as a biopsy needle or an electrode. At its proximal end, the shaft can be detachably connected or permanently fixed (mechanically and/or electrically) to a handpiece of the medical imaging device. In its distal end section, the shaft may include a closable opening through which the carrier device with the pre-mounted sensor assembly can be inserted into the interior of the shaft to ultimately be mounted therein. The opening in the end section of the shaft can be sealed by means of a cover, preferably providing a fluid-tight seal. Of course, the opening in the shaft can also be located at any other point in the shaft, for example in its center or its proximal end section.

The “longitudinal direction” of the shaft is a direction of a longest extension of the shaft. Accordingly, the “longitudinal axis” of the shaft is the axis that corresponds to the direction of its longest extension.

“Distal side” and “distal” are understood to mean an arrangement close to the patient's body and thus remote from the user and a corresponding end or section. Correspondingly, “proximal side” or “proximal” is understood to mean an arrangement close to and thus remote from the patient's body and a corresponding end or section. Thus, the “proximal direction” is a direction directed towards the proximal end of the medical imaging device and/or the handpiece and/or the endoscope. Accordingly, the “distal direction” is the opposite direction directed towards a tip and/or end of the shaft.

A “sensor” is a technical component that detects certain physical or chemical properties and/or the material properties of its environment either qualitatively or quantitatively as a measured variable. In principle, a sensors may detect or measure any physical or chemical property or properties, such as mechanical, thermoelectric, resistive, piezoelectric, capacitive, inductive, optical, acoustic or magnetic. For example, the sensor can be an acceleration sensor or a pressure sensor. For the purposes of this disclosure, the sensor is frequently an optical sensor and/or an image sensor, but this is not limiting.

An “image sensor” is a light-sensitive electronic component based on an internal photoelectric effect. The image sensor can be used to record one or more images from the viewing area of the medical imaging device and convert them into electronic signals. The image sensor has a sensor plane that is preferentially positioned in the image plane of the optical system, the lens system and/or the objective of the medical imaging device. The image sensor may be an electronic image sensor can be a CCD sensor (charge-coupled device) or a CMOS sensor (complementary metal oxide semiconductor). Preferably, the electronic image sensor is arranged directly as a chip in the distal end section and/or the tip of the shaft of the medical imaging device and/or the video endoscope and transmits the digital image signals from the distal end of the shaft to the proximal end by means of electrical transmission lines.

A “light source” is a component or device from which light is emitted. The light source can be designed as a stand-alone device, whereby light is coupled into the shaft and up to its distal end via a connection port and a light guide. The light source can also be an LED light source and/or a cold light source. The light source can be integrated directly into the medical imaging device, for example in the handpiece or in the shaft. Preferably, the light source is an LED or another light-generating device positioned directly in the distal end section of the shaft and/or the medical imaging device. In some embodiments, the light from the light source may be used to homogeneously illuminate the examination area and the desired viewing area.

A “viewing area” (sometimes referred to as the field of view) is an area, a volume, an outer surface and/or an inner surface of a human or animal body or a technical object. An optical image is generated from the entire viewing area or a part of the viewing area by means of a lens and/or captured by means of the image sensor. A viewing area can be, for example, an external skin surface, an internal organ, a natural cavity in a human or animal body, or the inside of a pipe.

The term “carrier device” refers to a device for pre-assembling, carrying and/or holding a sensor assembly. In particular, the carrier device can be a component or a component group. The carrier device can be designed in one or more parts. In principle, the carrier device can have any shape. However, the carrier device usually has external dimensions that are smaller than the dimensions of the cavity within the shaft in which the carrier device and/or the pre-mounted sensor assembly is or are to be accommodated. The carrier device may be designed with a greater length in the longitudinal direction of the shaft than its cross-section, and it may have a length in the range from 1.0 mm to 8.0 mm, preferably from 2.0 mm to 4.0 mm. The carrier device may have a width in the range from 1.0 mm to 4.0 mm, preferably from 2.5 mm to 3.5 mm. The carrier device may have a height in the range from 1.0 mm to 4.0 mm, preferably in the range from 1.5 mm to 2.5 mm.

A “side” of the carrier device is understood to mean both a surface and a directional indication. Since the carrier device can in principle have any external shape, such as completely or partially rectangular, round, cylindrical and the like, a side is not intended to necessarily have to be a clearly defined side with edges. The adjacent sides on the outer surface of the carrier device can also merge into one another, for example at a rounded juncture. Therefore, a side is also understood to be a surface or a surface section of the carrier device that is aligned in a certain direction. Thus, the upper side is usually aligned upwards, the lower side downwards, the two opposing sides to the right and left, and the distal side is aligned forwards or after installation in the shaft towards the distal end of the shaft, and correspondingly the proximal side is aligned to the rear or in the installed state towards the proximal end of the shaft. The respective side of the carrier device can be flat, inclined, or conically widening or narrowing in one direction. The distal side of the carrier device, on which the sensor and/or image sensor is preferably mounted or arranged, can be inclined. In some preferred embodiments the distal side runs diagonally upwards from the underside in a proximal direction to the upper side. In principle, the carrier device can be made of any type of material. For example, the carrier device can be made of plastic and/or metal. In addition to stainless steel, the carrier device can in particular have copper and/or brass in order to enable heat dissipation through the carrier device itself. The carrier device has at least one cavity running from its proximal side to its distal side. However, the carrier device can also have two or more continuous cavities and/or channels. However, in addition to and/or instead of the at least one continuous cavity, the carrier device can also have a continuous recess in the longitudinal direction on its outer surface.

A “cavity” is an empty and/or recessed space, at least before pre-assembly, inside the carrier device. After pre-assembly of the carrier device, an alignment tool and/or another component may be completely or partially arranged in the continuous cavity of the carrier device. One or more heat transfer elements can also be arranged in the cavity of the carrier device. The cavity may be arranged concentrically to the longitudinal center axis of the carrier device and/or the shaft. However, the continuous cavity or several continuous cavities can also be arranged eccentrically to the longitudinal center axis of the carrier device and/or the shaft. The cavity can be of any shape, for example rod-shaped with a rectangular or round cross-section. The cavity can be a continuous bore. The cavity may have a diameter in the range from 0.1 mm to 3.0 mm, preferably from 0.5 mm to 2.0 mm.

A “sensor assembly” is an object consisting of two or more parts or assemblies. The sensor assembly includes at least one sensor. The sensor assembly can be mounted and/or dismounted, and the individual components of the sensor assembly can be mounted and/or arranged on, on and/or in the carrier device, such that in the pre-assembled state the sensor assembly and the carrier device are present as a common assembly (also referred to as an assembled pre-assembly).

The term “alignment tool” refers to any type of tool that can be used to adjust the position and/or orientation of the sensor on the carrier device. An alignment tool can be a mandrel or a hook, for example. The alignment tool may be inserted into the cavity through the proximal opening of the continuous cavity and acts with its distal end on the back of the sensor and/or the sensor holder. For this purpose, the sensor and/or the sensor holder can have corresponding mating receptacles and/or recesses on their respective rear side and thus on its side aligned in the proximal direction, into which the tip of the alignment tool and/or mandrel can engage.

In one embodiment of the carrier device, the sensor assembly and/or the carrier device includes the sensor, a lens mount, a lens, and a circuit board.

Depending on the type of sensor and the components required, the sensor assembly can be pre-assembled alone and/or directly on the carrier device.

An “objective” is a light collecting optical system that produces a real optical image of an object and/or field of view. The objective includes at least one lens. Furthermore, the objective can also have two or more lenses and an aperture. On the distal side, the objective can be arranged behind a cover glass, whereby the cover glass adjoins the shaft at the distal end, or the cover glass can also be part of the objective. The objective can be an ultra-short objective. Preferably, the objective is arranged directly behind the cover glass and attached with its proximal side to the distal side of an image sensor. The objective can also be arranged in two or more parts and its elements may be distributed, for example on the distal side and the proximal side of the carrier device.

The “lens mount” is in particular a housing and/or a holder for the objective. In particular, the lens mount partially or completely surrounds the optical components of the objective. The lens mount may also be understood to be the location at which the objective is connected to another component, such as the cover glass or the sensor.

A “circuit board” may be any device which may carry electronic components such as a printed circuit board, a flexible circuit board and/or a control board. The circuit board includes electrical connections for the sensor and/or other components of the sensor assembly which are to be supplied with voltage. The circuit board is used in particular both for mechanical fastening and for electrically connecting the components of the sensor assembly in the assembled state. The circuit board is adhered to one side or the section of one side, preferably to the top or bottom, of the carrier device.

In order to enhance the use of the limited space in the cavity of the distal end section of the shaft and to enable an electrical connection between the proximal side of the carrier device and the handpiece of the imaging device as well as a power supply to the arranged sensor, the circuit board is arranged on the upper side or the underside of the carrier device. In addition, this arrangement on the upper or lower side of the carrier device permits the circuit board to not be in the optical path, such that the objective can also be arranged separately on both sides of the distal side and the proximal side of the carrier device and, accordingly, an image sensor is then arranged proximally to the proximal part of the objective.

In a further embodiment, the carrier device can have at least one heat transfer element or is designed as a heat transfer element. In order to achieve enhance heat dissipation and/or distribution, the at least one heat transfer element can be arranged at least partially in the cavity so that operating heat from the sensor can be dissipated in a proximal direction. The “heat transfer element” is a thermally conductive element, component, and/or a thermally conductive material. The heat transfer element and/or the carrier device itself may include a heat-conducting material, such as metal. The heat transfer element can also be a heat sink or a cooling element. For example, a copper disc may be arranged as a cooling element proximal to the proximal opening of the continuous cavity and the continuous cavity may be filled with air. The heat transfer element can also be designed as a copper wire or rod that is arranged in the cavity in the longitudinal direction and contacts the rear of the sensor and/or the sensor holder on the distal side. The heat transfer element can also be a copper tube that is arranged in the cavity of the carrier device and is itself filled with air. The cavity of the carrier device can also be filled with a heat-conducting paste after assembly. The heat transfer element may be coupled directly to a heat-emitting component, such as the sensor, a light source and/or another electronic component, via one of its contact surfaces. Heat can also be transferred and/or coupled indirectly via another component, the air-filled cavity of the carrier device, and/or a heat-conducting paste. The heat transfer element transfers the absorbed heat further in a proximal direction towards a heat conducting body such as a heat sink. The heat conducting body can be a heat pipe, for example. In particular, the heat pipe is arranged and extends in the proximal direction along the longitudinal axis of the shaft. The heat transfer element can be thermally coupled directly or indirectly with the heat conducting body.

In particular, “operating heat” refers to the heat or amount of heat that is generated due to the operation of the sensor, image sensor, light source, and/or another component causing an increase in temperature. The operating heat is generated, for example, by a power loss of the sensor and/or the light source and is emitted to the environment due to a temperature gradient.

In a further embodiment, the carrier device can have at least one recess in at least one side of the respective sides for receiving an adhesive, so that the sensor assembly can be pre-mounted on the carrier device outside the shaft and can then be inserted into the cavity of the shaft and bonded. This enables the carrier device with the pre-assembled sensor group to be inserted into the shaft as a single unit and then bonded to an inner wall of the shaft by means of the at least one recess and an adhesive, thereby fixing it in place.

In order to provide a defined attachment of the carrier device with the pre-mounted sensor assembly and a defined amount of adhesive, the carrier device can have two recesses, three recesses, four recesses and, optionally, further recesses. The positions and the respective design of the recesses determine both the respective bonding location and the quantity of adhesive, thereby simplifying the process, and avoiding the unwanted discharge of the adhesive into other areas of the shaft cavity. As a result, both the connection of the carrier device within the shaft and the dispensing of the adhesive are improved. The recess or recesses provide a defined reservoir of the adhesive, which ensures bonding without smearing within the shaft. Preferably, the adhesive is introduced into the respective recess while the shaft is still open, for example by means of a cannula, after the carrier device with the pre-mounted sensor assembly has been inserted into the intended position within the cavity of the shaft. However, as an alternative to gluing, the carrier device can be molded into the interior of the shaft by means of the recess or recesses. In some embodiments of the carrier device, the recesses are arranged on opposite sides. Thus, the recesses in the inserted carrier device may be arranged laterally to the left and right in the shaft, aligned in the distal direction, and the carrier device is bonded laterally.

In order to further improve the connection between the carrier device and an inner surface of the shaft and/or to enable force transmission or absorption, a rib can be arranged between two recesses, whereby an inner surface of the shaft can be contacted by means of an outer surface of the rib. This provides a defined contact surface of the carrier device to the inner surface of the shaft and/or a defined bonding surface via one or more ribs.

A “recess” is a recessed space on and/or in the outer surface of the carrier device. In particular, a recess is an incision and thus a depression in the outer surface of the carrier device. The recess and/or the recesses can be arranged on each side of the carrier device and/or on each section of its outer surface. Preferably, the recess or recesses are arranged on the two opposite sides and thus on the left and right sides of the carrier device when aligned in the distal direction. The recess or recesses may be formed in a longitudinal direction from the top side to the bottom side. The recess does not necessarily have to be continuous in its longitudinal direction from the top to the bottom. For example, the recess can also be closed on the upper side and/or the lower side and only open laterally towards the inner surface of the shaft. The closed upper side or underside can also have a small through-opening so that the adhesive can be introduced into the recess through this small through-opening using a cannula, for example. The recess can have any shape. Likewise, two or more recesses can have the same shapes, dimensions and/or orientations or correspondingly different orientations, shapes and/or dimensions. The recess and/or the recesses can or can have any shape, in particular in cross-section, such as rectangular or semi-circular. The respective recess can, for example, be produced by milling into the outer surface of the carrier device. A rib of the carrier device can simply be the raised area between two recesses or be specially formed. The rib may have a defined outer surface and/or contact surface which can contact an inner surface of the shaft and/or be bonded to it. Both the surface of the respective recess and the outer surface of the rib can be smooth, rough and/or textured. These surfaces can also be partially smooth, rough and/or textured. Structural elements can also be arranged on the surface of the recess, for example towards its edges, which increasingly extend into the recess and thus prevent the adhesive from flowing away. By structuring the outer surface of the recess in this way, and/or by means of a respective rib, separate adhesive pockets can be provided, between which smearing and/or transfer of the adhesive is prevented.

An “adhesive” (also known as a glue) is usually a non-metallic substance with which the materials of the carrier device and the shaft are bonded by surface adhesion and by means of its internal strength. Any adhesive that enables the respective materials of the carrier device and the shaft to be bonded is suitable. In particular, the adhesive is used to create a material bond between the carrier device and the inner surface of the shaft. In order to prevent unwanted reflections and thus contaminations to captured image, the adhesive can be colored black. The adhesive can be a light-cured adhesive (also known as UV cured adhesive), for example, which enables the connection to cure quickly.

Also presented is medical imaging device, in particular a video endoscope with a handpiece and an elongated shaft, wherein the medical imaging device has a light source for illuminating a viewing area and the elongated shaft has a cavity, an electrical conductor and/or a working channel, wherein the medical imaging device has a previously described carrier device, and the sensor is designed as an electronic image sensor for capturing images of the viewing area. The sensor/sensor group can be pre-assembled on the carrier device outside of the shaft and the functionality of the image sensor can be tested outside the device itself in the pre-assembled state. This permits any malfunctions of the mounted image sensor to be detectable at an early stage before the mounted image sensor is finally fixed inside the narrow shaft. This simplifies assembly and is more cost-effective. Additionally, any non-functional image sensor and/or one that is not mounted in accordance with the specification can be detected and replaced outside the video endoscope, such that the entire video endoscope does not have to be disposed of when a non-functioning or incorrectly aligned the image sensor is subsequently detected.

In order to be able to easily insert the carrier device with the pre-mounted sensor assembly as one component into the shaft, the shaft can have a closable opening for inserting the carrier device and/or the sensor assembly. In particular, the opening may have at least slightly larger dimensions than the outer dimensions of the carrier device with the pre-mounted sensor assembly. The opening may be closed by means of a cover. The cover may also be fixed to a contact surface around the opening by means of an adhesive or by means of the same adhesive used for the recess. The cover can also be welded.

In a further embodiment of the medical imaging device, at least one heat conducting tube is arranged in the shaft in a distal direction in such a way that the at least one heat transfer element of the carrier device is coupled to the heat conducting body in a heat-transferring manner. Thereby, the heat absorbed by the heat transfer element can be removed and/or dissipated in a targeted manner by means of the heat conducting body. Direct thermal coupling between the heat transfer element and the heat conducting body can be achieved through a common contact surface. There can also be indirect heat coupling, for example by a heat-conducting paste arranged between the proximal end of the heat transfer element and the distal end of the heat conducting body.

Also presented is a method for pre-mounting a sensor assembly on a carrier device and for insertion into a shaft of a medical imaging device, wherein the sensor assembly comprises at least one sensor, the carrier device comprises as a respective side a distal side, a proximal side, a top side, a bottom side and two opposing sides, and the carrier device comprising a continuous cavity from the proximal side to the distal side. The method includes connecting a lens to the sensor so that the lens is positioned distal to the sensor; connecting the sensor to a distal side of the carrier device; optionally inserting an alignment tool into the cavity of the carrier device in a distal direction and aligning the sensor from the inside to the distal side of the carrier device by means of an alignment tool; and inserting the carrier device into the shaft of the medical imaging device.

Thus, a method is provided by which a sensor assembly with at least one sensor is pre-assembled in, on and/or at the carrier device in a simple and cost-effective manner and subsequently inserted into the shaft of the medical imaging device as a pre-assembled pre-assembly. The method is carried out with a previously described carrier device and/or a previously described medical imaging device.

In a further embodiment of the method, before insertion into the shaft, the circuit board is connected to an upper side or lower side of the carrier device, and/or a heat transfer element is inserted at least partially into the cavity of the carrier device. In order to arrange and fasten the pre-assembled preassembly and/or the carrier device with the sensor assembly in a defined manner in the cavity of the shaft, an adhesive is introduced into at least one recess in one side of the carrier device before or after insertion, so that the carrier device is bonded to an inner side of the shaft.

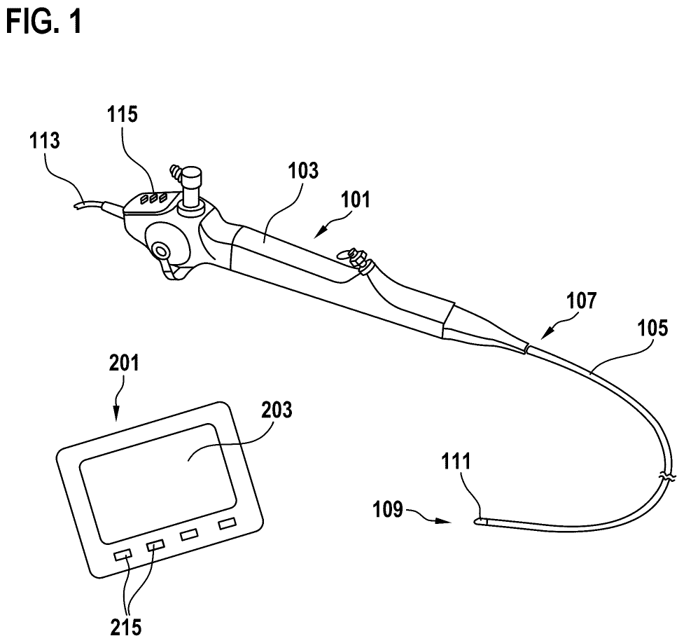

Turning now to the particular embodiments shown in the figures, FIG. 1 shows a video endoscope 101 comprising a handle 103 and an elongate shaft 105 connected together at a proximal end 107 of the shaft 105. The handle 103 includes controls 115 and is connected via a cable 113 at its proximal end to an external control and processing unit such as a camera control unit (CCU), not shown, and to a display 201. The display 201 includes a monitor 203 for displaying endoscopic images and controls 215.

The video endoscope 101 is designed to provide video and image data from an viewing area within a cavity of a body. The shaft 105 comprises a distal end section 111 at its distal end 109.

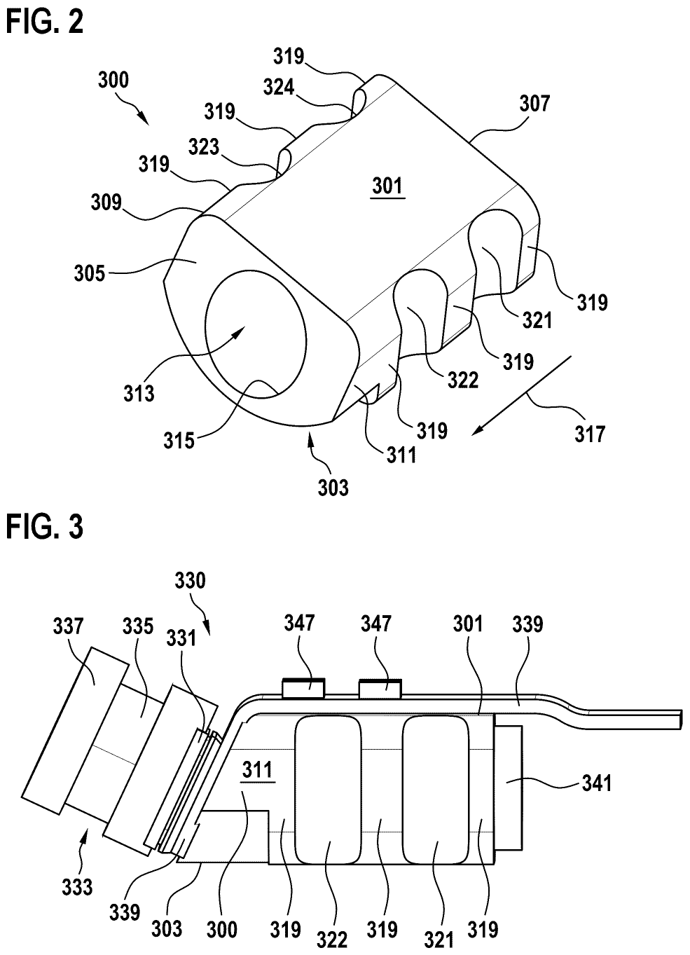

FIGS. 2-5 illustrate embodiments where the carrier device is a sensor holder 300 that has a top side 301 and an opposite bottom side 303. Viewed in a distal direction 317, the sensor holder 300 has a proximal side 307 and an opposite distal side 305 as well as a right side 309 and a left side 311. From the proximal side 307 to the distal side 305, a continuous cavity 313 is arranged on the inside of the sensor holder 300. The cavity 313 is arranged with its circular cross-section concentric to a longitudinal center axis of the sensor holder 300. As an example, the cross-section of the cavity 313 shown has a diameter of 1.6 mm. A width of the distal side 305 is 2.85 mm. A length of the sensor holder 300 along the bottom side 303 is 3.0 mm. A height of the sensor holder 300 is 2.0 mm. The distal side 305 is arranged obliquely to a transverse plane perpendicular to the longitudinal axis of the sensor holder 300 so that the top side 301 of the oblique distal side 305 is set back in the proximal direction (opposite the distal direction 317). All of the measurements set forth in this exemplary embodiment are not limiting.

On the right side 309 and the left side 311, the sensor holder 300 has three ribs 319 each, with a first recess 321 and a second recess 322 being arranged between the ribs 319 on the left side 311 and a third recess 323 and a fourth recess 324 being arranged on the right side 309 (FIG. 2).

In a mounted state, a sensor assembly 330 is arranged on the sensor holder 300 (FIG. 3). The sensor assembly 330 has an electronic image sensor 331, an objective 333 with an objective mount 335, and a circuit board 339. A cover glass 337 is arranged on the distal side of the objective mount 335. In the assembled state, a heat sink 341 in the form of a copper wire is inserted into the cavity 313 of the sensor holder 300, which abuts the rear of the image sensor 331 on the distal side.

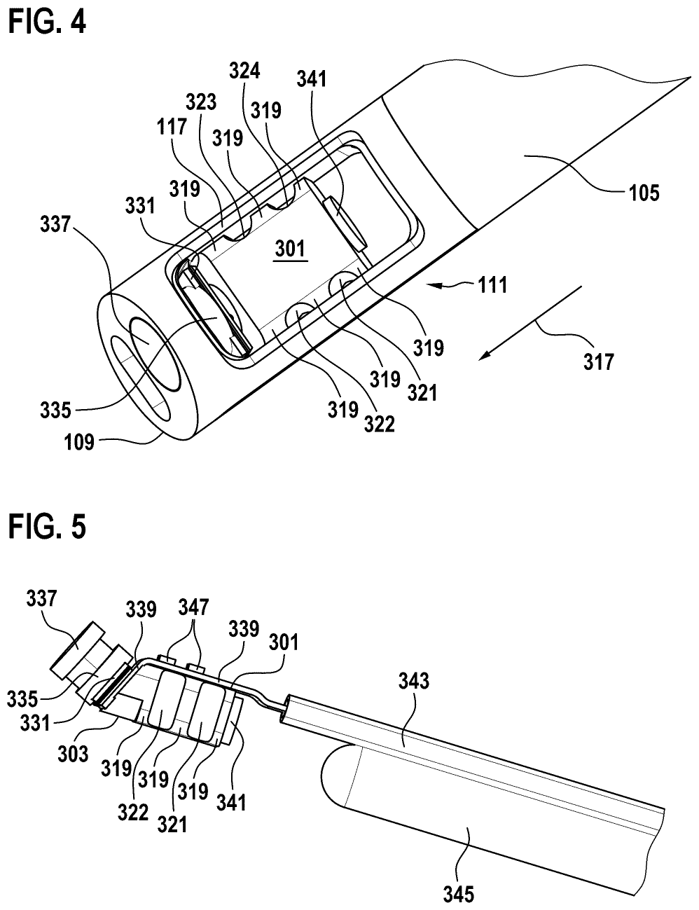

As seen in FIG. 4, the pre-assembled sensor group 330 on the sensor holder 300 is shown inserted into the cavity 313 of the shaft 105 in the distal end section 111 through the mounting opening 117. For display reasons, the upper side 301 of the sensor holder 300 is visible, but not the circuit board 339 which would normally be mounted thereon.

The circuit board 339 has several electronic components 374 on its upper side and is electrically connected proximally to the handle 103 of the video endoscope 101 in a connecting tube 343. A heat conducting pipe 345 is arranged below the connecting tube 343. A heat-conducting paste (not shown) is arranged between the distal end of the heat conducting pipe 345 and the proximal end of the heat sink 341.

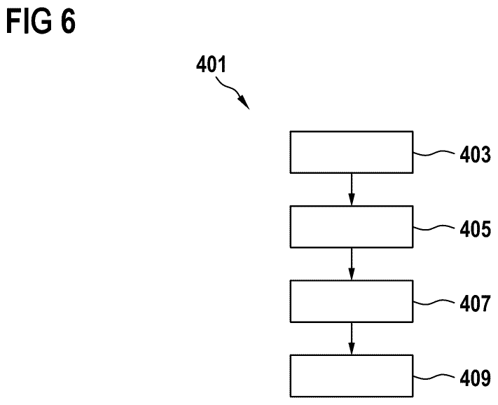

FIG. 6 shows a flowchart for a method 400 for pre-assembling the sensor assembly 330. Outside the video endoscope 101, the objective 333 is bonded to the distal side of the image sensor 331 (step 401). The image sensor 331 is then connected to the proximal side of the objective 333 side by bonding to the distal side 305 of the sensor holder 300 (step 403). Subsequently, before the adhesive bond is cured, an alignment tool, such as a mandrel, is inserted from the proximal side 307 through the cavity 313 of the sensor holder 300 and the image sensor 331 is aligned from the inside by means of the mandrel (step 405). Subsequently, the image sensor 331 pre-mounted in this way on the sensor holder 300 and the lens 333 are tested for functionality, preferably at the assembly site, such as the factory prior to shipment to a customer. After a positive test, the heat sink 341 is inserted into the cavity 313 through the proximal opening of the cavity 313 so that the heat sink 341 contacts a rear side of the image sensor 331. Subsequently, the sensor holder 300 with the pre-mounted sensor assembly 330 is inserted into the cavity of the shaft 305 through the mounting opening 117 (step 407). During insertion, the pre-assembled sensor assembly 330 is displaced in the sensor holder 300 in the distal direction 317 until the cover glass 337 is inserted into a provided receptacle at the distal end 109 of the shaft 105 and is bonded there (FIG. 4). By means of, for example, a cannula, a UV adhesive is subsequently introduced from above into the first recess 321, the second recess 322, the third recess 323, and the fourth recess 324 so that the region of the recesses 321 to 324 and the outer sides of the ribs 319 are bonded with the inner surface of the shaft 105 (step 409). Subsequently, a cover (not shown) is adhered, such as by welding, to the assembly opening 117 such that the shaft 105 is sealed in a fluid-tight manner at its distal end section 111.

During subsequent operation of the video endoscope 101, the operating heat emitted by the image sensor 331 is transferred to the proximal side of the heat sink 341 arranged within the sensor holder 300 by direct contact with the distal side of the heat sink 341. There, the heat sink 341 is in contact with thermally conductive paste, which is arranged between the heat sink 341 and the heat conducting pipe 345, such that the heat is further transferred to the heat conducting pipe 345 via the thermally conductive paste and dissipated in the proximal direction.

Thus, a sensor holder 300 is provided which enables improved pre-assembly of the sensor assembly 330 and alignment and functional testing of the image sensor 331 outside the shaft 105. This and other advantages of the embodiments and methods disclosed herein provide a multifunctional sensor holder 300, which can be used advantageously both during pre-assembly and during operation of the video endoscope 101.

The foregoing has outlined rather broadly the features and technical advantages of the invention in order that the detailed description of the invention that follows may be better understood. It should be appreciated by those skilled in the art that the conception and specific embodiments disclosed may be readily utilized as a basis for modifying or designing other structures for carrying out the same purposes of the invention. It should also be realized by those skilled in the art that such equivalent constructions do not depart from the scope of the invention as set forth in the appended claims.

Although the invention and its advantages have been described in detail, it should be understood that various changes, substitutions and alterations can be made herein without departing from the scope of the invention as defined by the appended claims. The combinations of features described herein should not be interpreted to be limiting, and the features herein may be used in any working combination or sub-combination according to the invention. This description should therefore be interpreted as providing written support, under U.S. patent law and any relevant foreign patent laws, for any working combination or some sub-combination of the features herein.

Moreover, the scope of the present application is not intended to be limited to the particular embodiments of the process, machine, manufacture, composition of matter, means, methods and steps described in the specification. As one of ordinary skill in the art will readily appreciate from the disclosure of the invention, processes, machines, manufacture, compositions of matter, means, methods, or steps, presently existing or later to be developed that perform substantially the same function or achieve substantially the same result as the corresponding embodiments described herein may be utilized according to the invention. Accordingly, the appended claims are intended to include within their scope such processes, machines, manufacture, compositions of matter, means, methods, or steps.

### LIST OF REFERENCE SYMBOLS

101 Video endoscope
103 Handle
105 Shaft
107 Proximal end of the shaft
109 Distal end of the shaft
111 Distal end section
113 Cable
115 Control
117 Mounting opening
201 Display
203 Monitor
215 Control
300 Sensor holder
301 Top side
303 Bottom side
305 Distal side
307 Proximal side
309 Right side
311 Left page
313 Cavity
315 Distal opening
317 Distal direction
319 Rib
321 First recess
322 Second recess
323 Third recess
324 Fourth recess
330 Sensor assembly
331 Image sensor
333 Objective
335 Objective mount
337 Cover glass
339 Circuit board
341 Heat sink
343 Connecting tube
345 Heat conducting pipe
347 Electronic component
400 Procedure for pre-assembling a sensor assembly
401 Connecting an objective to the image sensor
403 Connecting the image sensor to a distal side of the sensor holder
405 Optional alignment of the image sensor
407 Inserting the sensor holder into the shaft
409 Bonding the sensor holder to the shaft

1. A carrier device for pre-mounting and holding a sensor assembly and for insertion into a shaft of a medical imaging device, the carrier device comprising a distal side, a proximal side, a top side, a bottom side, and two opposing sides, wherein the sensor assembly is associated with the carrier device, the sensor assembly comprising at least one sensor, a bottom side and two opposing sides, wherein the sensor is configured to be mounted on the distal side of the carrier device, and wherein the carrier device has a continuous cavity from the proximal side to the distal side, and is configured such that when the sensor is preassembled on the distal side of the carrier device, the sensor can be aligned from the inside of the carrier device into a predetermined position by means of an alignment tool which can be inserted into the cavity.

2. The carrier device of claim 1, wherein the sensor assembly and/or the carrier device comprises the sensor, an objective mount, an objective, or a circuit board for electronic components.

3. The carrier device of claim 2, wherein the circuit board is arranged on the top side or the bottom side of the carrier device.

4. The carrier device of claim 2, wherein the carrier device further comprises at least one heat transfer element or is itself configured as a heat transfer element.

5. The carrier device of claim 4, wherein the at least one heat transfer element is positioned at least partially in the cavity and is configured such that an operating heat of the sensor is dissipated in a proximal direction.

6. The carrier device of claim 1, wherein the carrier device further comprises at least one recess in at least one side for receiving an adhesive, such that the sensor assembly can be pre-mounted on the carrier device outside the shaft and then be inserted into the cavity of the shaft and be adhesively bonded thereto.

7. The carrier device of claim 6, wherein the carrier device comprises a plurality of said recesses.

8. The carrier device of claim 7, wherein a number of the plurality of recesses are arranged on the opposite sides to a respective opposing number of recesses.

9. The carrier device of claim 7, further comprising a rib arranged between two recesses on a given side, and wherein an inner surface of the shaft is contactable by means of an outer surface of the rib.

10. The carrier device of claim 8, further comprising a rib arranged between two recesses on a given side, and wherein an inner surface of the shaft is contactable by means of an outer surface of the rib.

11. The medical imaging device of claim 1, wherein the sensor is an image sensor.

12. A medical imaging device, comprising a handpiece, an elongated shaft, a light source positioned at a distal end section of the shaft for illuminating a viewing area, wherein the elongated shaft comprises a cavity, an electrical conductor and/or a working channel, and wherein the medical imaging device further comprises a carrier device for pre-mounting and holding a sensor assembly and for insertion into the cavity of the shaft, the carrier device comprising a distal side, a proximal side, a top side, a bottom side, and two opposing sides, wherein the sensor assembly is associated with the carrier device, the sensor assembly comprising at least one sensor, a bottom side and two opposing sides, wherein the sensor is configured to be mounted on the distal side of the carrier device, and wherein the carrier device has a continuous cavity from the proximal side to the distal side, and is configured such that when the sensor is preassembled on the distal side of the carrier device, the sensor can be aligned from the inside of the carrier device into a predetermined position by means of an alignment tool which can be inserted into the cavity.

13. The medical imaging device of claim 12, wherein the sensor is an image sensor.

14. The medical imaging device of claim 12, wherein the medical imaging device is a video endoscope.

15. The medical imaging device of claim 12, wherein the shaft comprises a closable opening configured for inserting the carrier device or the sensor assembly thereinto.

16. The medical imaging device of claim 12, wherein at least one heat-conducting body is arranged in the shaft and aligned in the distal direction configured such that the at least one heat-transfer element of the carrier device is thermally coupled to the heat-conducting body.

17. The medical imaging device of claim 15, wherein at least one heat-conducting body is arranged in the shaft and aligned in the distal direction configured such that the at least one heat-transfer element of the carrier device is thermally coupled to the heat-conducting body.

18. A method of pre-mounting a sensor assembly on a carrier device and inserting it into a shaft of a medical imaging device, the sensor assembly having at least one sensor, the carrier device comprising a distal side, a proximal side, a top side, a bottom side and two opposing sides, and the carrier further comprising a continuous cavity from the proximal side to the distal side, comprising the steps of:
connecting a lens to the sensor so that the lens is positioned distal to the sensor;
connecting the sensor to a distal side of the carrier device;
optionally, inserting an alignment tool into the cavity of the carrier device in a distal direction and aligning the sensor from the inside to the distal side of the carrier device by means of an alignment tool, and
inserting the carrier device into the shaft of the medical imaging device.

19. The method of claim 18, comprising the further step of, prior to the insertion into the shaft, connecting a circuit board to an upper side or lower side of the carrier device, or inserting a heat transfer element at least partially into the cavity of the carrier device.

20. The method of claim 18, comprising the further step of introducing an adhesive into at least one recess in one side of the carrier device, such that the carrier device is bonded to an inner side of the shaft.
